(19) United States
(12) Patent Application Publication
(10) Pub. No.: US 2016/0378938 A1
(43) Pub. Date: Dec. 29, 2016

(54) CONTACTLESS DEVICE CONTROL SYSTEM IN STERILE MEDICAL ENVIRONMENT

(71) Applicant: Siemens Healthcare GmbH, Erlangen (DE)

(72) Inventors: Soeren Kuhrt, Erlangen (DE); Bastian Rackow, Erlangen (DE); Stefan Reichelt, Bamberg (DE)

(73) Assignee: Siemens Healthcare GmbH, Erlangen (DE)

(21) Appl. No.: 15/190,560

(22) Filed: Jun. 23, 2016

(30) Foreign Application Priority Data
Jun. 26, 2015 (DE) 102015211965.3

(51) Int. Cl. G06F 19/00; G05B 15/02; G05B 19/07; A61B 5/11; A61B 5/00

(52) U.S. Cl. CPC G06F 19/3406; A61B 5/11; A61B 5/742; G05B 19/07; G05B 15/02; A61B 2562/0219; G05B 2219/37432; G05B 2219/45123

(57) ABSTRACT

In a control system and method for controlling a medical device while observing sterile conditions, a portable controller is provided that has at least one inertial sensor to acquire acceleration data for a body part of a user. The portable controller further has a wireless interface for the transmission of the acquired acceleration data to a conversion module. The conversion module receives the transmitted acceleration data and converts it into instructions, and the instructions are used to control the medical device.

### BACKGROUND OF THE INVENTION

Field of the Invention

The present invention concerns medical technology and physics together with information technology and in particular concerns the control of a medical system by the use of specific inertial sensors.

Description of the Prior Art

In the field of medicine, the observance of sterile conditions is extremely important in order to keep the risk of infection during medical interventions and in the surgical environment as low as possible.

With modern working practices, it is has become usual, even during an intervention or immediately prior thereto, to refer to radiological image data acquired or provided by, for example a computed tomography system. To operate the technical medical system or device, the system or the device has to be controlled by user inputs.

To this end, two methods are used in the prior art for controlling an image display device for radiological images.

In the one method, the operation of the device is delegated to an assistant so that it is not the actual doctor who operates the device in the sterile environment. The doctor only gives the assistant instructions as to the operation of the device. This method is susceptible to errors since it is possible for instructions to be misunderstood, and it is uneconomical and slow.

The other basic known method achieves the observance of requirements in the sterile environment a sterile covering, for example in the form of a hood, being placed over the respective operating element (joystick, mouse, touchscreen etc.) through which the operating element is then operated. However, this procedure has the disadvantage that operability is very limited and there is still a risk of contamination.

It is desirable to achieve unequivocal and contactless operation of computer-based devices and functions, such as the operation of an image display of radiological images or the control of other device functions.

Also known in the prior art is the use of gesture-based systems, with which a gesture made by a user is used to control the system is optically acquired. However, in tests, this technique does not produce satisfactory results. Currently known gesture-based systems (for example LeapMotion, Kinect) operate with optical recognition and camera scanning. These systems are optimized with respect to a specific distance from the user, or defined gestures, and in practice have been found to be insufficiently robust for applications outside the consumer field. One significant disadvantage is the restricted field of view or the possible visible coverage of the device, which is not acceptable in the medical environment. This greatly restricts the user's freedom of action. In addition, depending on the technology used, the detection accuracy can be impaired depending upon the illumination conditions, reflections etc.

### SUMMARY OF THE INVENTION

An object of the present invention is to facilitate safe and reliable control of a technical medical system even in a sterile environment. Great emphasis is in particular placed on the extraordinarily important functional robustness in the field of medicine and on intuitive operation thus avoiding long learning curves.

The following describes the achievement of the object with reference to the inventive control system. All features, advantages and/or alternative embodiments mentioned herein are applicable to all aspects of the invention. In other words, the system and the storage medium can also be developed with the features described in conjunction with the method. The functional features of the method are embodied by corresponding substantive computer-implemented modules, in particular microprocessor modules in the system. The control system and the method also can be integrated as embedded systems in the medical system or in the system as a whole.

According to one aspect of the invention, the above object is achieved by a control system for controlling a computer-operated technical medical device that has to be operated under sterile conditions. To this end, the control system has a controller, which can be carried by the user in the sterile environment that has at least one inertial sensor that acquires acceleration data of a body part of the user. The portable controller further has a wireless interface for transferring the acquired acceleration data to a conversion module. The conversion module is a component of the control system and receives the transmitted acceleration data and converts the data into instructions, with the instructions in a form (formatted) for use to control the medical device.

The terms used herein are explained in more detail below.

The computer-operated technical medical device is a device with at least one interface to a computer. This is preferably an image display device, such as a monitor, for the display of radiological data and images, in particular in DICOM format (DICOM: Digital Communication in Medicine) optionally with a control component, such as a graphics card or a software-based control system. Hence, the device can have a monitor with a control processor (for example in the form of a graphics card). The image data displayed on the monitor must be manipulated such as being selected, shrunk, enlarged, rotated or modified in some other way. This requires user inputs in order to initiate and execute the necessary modifications so that a new graphical display can be presented on the monitor in response to the user gesture.

According to the invention, the user inputs are acquired exclusively in a contactless manner. The contactless control of the device by the use of user inputs is performed by at least one inertial sensor with which the acceleration data of a body part of the user (for example a movement of a hand, arm or finger) are acquired. In a preferred embodiment of the invention, all user interactions or all user inputs are acquired exclusively via the portable controller. Advantageously, the body movements or gestures according to the invention are not acquired optically, but rather acceleration or gyro sensors are used to determine exact position and acceleration data, which are automatically converted into predefined instructions.

As mentioned above, the primary embodiment of the invention relates to the control of an image-display process and the associated user inputs for controlling the image display. However, the contactless control system can also be applied to other computer-based processes that have to be controlled under sterile conditions, such as the control of other software or hardware programs, such as the control of imaging per se or processes associated therewith. However, the contactless control system according to the invention should primarily be used to control a display process. The control of the display process usually requires the operation of a pushbutton on a user interface. Since the methods known from the prior art for the operation of pushbuttons cannot be used in view of the requirements with respect to sterility, these pushbutton(s) are exclusively operated in a contactless manner by means of the acquisition of gestures detected by the inertial sensors.

In the operating theater and in particular during an operation, a sterile environment or sterile zone is established in order to prevent exposure of the patient to bacteria to the greatest degree possible. The sterile zone, which must not be entered by non-sterile staff, contains the patient covered with sterile (germ-free) drapes, the instrument table and the environment of the surgeon and assistants wearing sterile clothing. However, it is now necessary for commands to be entered as inputs to control the device or the system (for example, the device for displaying the radiological data) in this sterile environment. According to the invention, to this end, a portable control unit is provided. The term “portable” means that the user is able to temporarily carry the controller on the user's body (i.e., it can be donned and removed as needed). The controller is embodied as a mobile electronic component and can be provided as a separate unit or integrated in a more complex component (for example a smartwatch etc.). To enable unrestricted freedom of movement, the controller only has wireless interfaces for forwarding the acceleration data acquired by the inertial sensor, which are provided as an output signal, and/or for reading in activation data as input data.

As input data, an activation signal and/or a deactivation signal, for example, can be sent to the control system. In this case, the activation signal can indicate that the contactless device control system should be activated for the duration of a predefinable time interval in order to avoid any unnecessary acquisition of acceleration data. Hence, this enables the provision of an ON- and an OFF-mode for the contactless device control system. In the case of a predefinable deactivation signal, the contactless device control system can be deactivated or even interrupted for a temporary period only.

Like the portable controller, the conversion module is an electronic component, in particular a microprocessor chip, which interacts with the software for controlling the device. Gestures are extracted or calculated from the acceleration data acquired by the inertial sensor. In a memory, each gesture can be assigned a user input and stored as a data tuple (overall in the form of a table). In addition, it is possible for multiple gestures to be executed for one user interaction. This assignment is defined by a preparatory definition phase, and can be changed again at a later time.

In principle, the extraction of the gestures from the raw data (for example acceleration data) and the extraction of the user interactions from the gestures take place in a freely programmable manner (i.e. not necessarily by the use of tables). The mode of operation of the conversion module can be described by means of the flowchart explained below.

1. The acceleration data are supplied to the conversion module by the controller (for example an armband).

2. The conversion module stores the data in a temporary buffer memory.

3. The current and stored data are forwarded to a processing unit.

4. The processor uses a program code provided to derive the gesture made from this data. It is also possible to refer to gestures made in the past to determine the gesture (self-learning system).

5. The identified gesture is sent to the control computer of the medical device.

6. The device initiates a user interaction as a function of the gesture and as a function of the current program context.

This is illustrated by the following example:

1. The armband sends the data item “High acceleration upward”

2. The conversion module stores this data item in the buffer memory.

3. The conversion module transfers the current data item and the last three data items to the processing unit. All four data items are worded “High acceleration upward”.

4. The processor derives the gesture “Swipe up” from the quadruple “High acceleration upward” data items.

5. The gesture “Swipe up” is sent to the control computer of the medical device.

6. The context of the medical device is “Selection menu open”. In this context and following the gesture “Swipe Up”, the processing unit initiates the interaction “Close menu”.

In a preferred embodiment of the invention, the inertial sensor is or includes an acceleration sensor and/or at least a gyroscope. In an acceleration sensor, accelerometers measure linear accelerations (preferably expressed in mV/g) respectively along one or more axes. Gyroscopes measure angular velocities (expressed in mV/°/s). If the above-described acceleration sensor is picked up and rotated about the longitudinal axis, the output of the module does not respond to changes to the angular velocity. In order to be able to acquire such changes as well, it is possible according to the invention for gyro sensors to be provided. This enables further gestures to be defined that (for example, even additionally to the movement) require a rotation of the respective body part of the user.

In principle, there are two different types of control functions and the respectively assigned user inputs.

A first type—the direct mode—is characterized by a user gesture being converted directly into a control signal for the computer program. This includes, for example, gestures for scrolling through a stack of images for the so-called “manipulation” of DICOM images or for the selection of a specific slice in a stack of slices with 3D images. As soon as this gesture is acquired, it can be directly converted and, for example, initiate a changed display on the screen.

In the second type—the confirmation mode—an instruction first has to be confirmed before it can be executed or converted. To this end, it is common to provide a keypad or button, which is displayed on a user interface, and the instruction is confirmed by a mouse or keystroke. Only when the confirmation signal has been entered in this way can the respective instruction be executed. Since the method according to the invention is executed in a contactless manner, it is not possible to initiate functions by a keystroke as is common in the prior art, for example on the mouse. Therefore, according to the invention, alternative methods are implemented for the acquisition of the confirmation signal.

In order to acquire the confirmation signal in a contactless manner, according to the invention a “Function by remaining” is implemented. In this case, a function is selected by the user causing the cursor to hover over a functional element (button, switch, menu etc.) for a certain time. Following the expiration of this time, for the confirmation a further functional element in the vicinity of the cursor is superimposed on the user interface, which the user must now touch with the cursor (here “touch” means “touch the functional element with the cursor on the screen”, i.e. no physical contact) or to hover thereover for a short period. After this, the function is deemed to be selected. This solves the problem of the function selection. The fact that the mouse cursor remains on a pushbutton causes the system automatically to switch to a selection mode permitting a defined function selection. This can be achieved either by the mouse cursor or by highlighting the corresponding function in a submenu.

Alternatively or in addition to the above-named method “Function by remaining”, it is possible to use the method “Function by gesture” for the confirmation mode.

Certain movements of the armband can be identified by the software as a gesture and initiate a function instead of changing the position of the cursor. Depending upon the embodiment of the invention, the following options can be implemented:

a) rapid movement to and fro in order to discard a selection or close an open menu;

b) rapid movement out of an environment in order to leave this environment without hereby changing the position selected within this environment (for example slice selection in a segment of an MPR view. If the segment is left with a rapid gesture, the slice selection set remains unchanged);

c) gentle rotation of the arm for the function selection (for example switching between manipulation of axial and coronal slices by rotating the arm);

d) encircling elements for selection;

e) strong movement to terminate ongoing functions.

According to an embodiment of the invention, additionally at least one vibration module is used in the control system to provide the user with feedback about the outcome of the respective operation (successful operation—mild vibration; faulty or unsuccessful operation—strong vibration).

When the control system is operated in the confirmation mode, following the acquisition of an instruction to be confirmed, a switching element is depicted on the display device, The switching element can be switched via a user gesture, preferably, but not necessarily, the user gesture is acquired via the inertial sensor and used to confirm the preceding instruction and initiate the conversion of the instruction. The switching element for the confirmation can be embodied as a mechanical switch (for example in form of a pedal switch or a lever) or as a voice-controlled switch (for example, activated by a voice message). In one advantageous embodiment of the invention, the mechanical switch implementation and the voice-controlled implementation can be combined. This enables flexible alternation between different modes for the user input (gesture-based, wherein the gestures are acquired with the inertial-sensor-based control unit) and the confirmation signal (mechanical input or voice input).

In another embodiment, a START gesture embodied to initiate the contactless control function is predefined. It is also possible for an END or termination gesture embodied for the termination of the contactless control function to be predefined. This should prevent “normal” movements of the user being acquired for the control system where no control is intended.

In order to achieve a high degree of flexibility, the portable controller is embodied as a modular component and includes the inertial sensors. The controller is preferably embodied in the form of a circlet or ring which can be pushed over sterile clothing onto a body part of the doctor (for example arm, finger or hand) in a rapid, simple and uncomplicated way as a sterilized element. The inertial sensors of the portable controller then acquire the movement of the arm, of finger or hand. Alternatively, the portable controller can be worn under the sterile clothing and can then also be used in a non-sterile condition. In another embodiment of the invention, the portable controller can be integrated in a further component, such as an armband or watch or other device.

Preferably, the controller and the conversion module are embodied as structurally separate components and interact via a wireless interface. However, the conversion module can also be integrated in the portable control unit. Similarly, the conversion module could also be implemented on a computer or a chip on which the program to be controlled (for example the image display) is executed.

The contactless control can take place at any spatial position. In contrast to methods known from the prior art, it is not necessary for the user to be located directly in front of the monitor with the user interface or in front of the joystick in order to be able to reach or operate the pushbutton displayed thereupon or the joystick. According to the invention, the contactless control is executed by the inertial sensors independently of the current position of the user. In addition, the control can take place when the user moves or changes position. This greatly increases the flexibility.

In a tested embodiment of the invention found to be preferable, the control system also has an RFID chip. This is used inter alia to identify the spatial position of the operator, for example for avoiding collision with C-arms. In a further embodiment of the invention the control system has a dosimetry probe. This is used for the acquisition of the critical operator radiation dose in this environment. The aforementioned embodiments of the invention can also be combined. It is possible for the operator to log onto the system with personalized modules in order to facilitate authentication. In addition, the personalized logon enables user-specific operator preferences to be automatically preset.

In a further, preferred embodiment of the invention, an auxiliary module is connected to the control system or integrated therein to indicate to the user that the working area been exited (loss of the radio connection). This can take place inter alia by vibration, in order to prevent the loss of the modules.

According to a further aspect, the invention encompasses a control method for contactless control of a technical medical or electronic device while observing sterile conditions, thus enabling the method to be used in the operating theater. The method has the following steps:
activation of a contactless device and/or application control (for example to display von medical images) in a sterile environment;
acquisition of acceleration data for a body part of a user;
transmission of the acquired acceleration data via a wireless interface to a conversion module;
reception of the acceleration data transmitted to the conversion module and the conversion of the acceleration data into instructions;
control of the device (for example the image-display process on an image display device) on the basis of the instructions.

In a preferred development of the inventive method, additionally or alternatively to acceleration, angular velocity is acquired by gyro sensors.

When the device has been controlled by the inertia-based input data for the calculation of the instructions, it is also possible for the result of the control to be presented as an output. If, for example, an image display process is to be controlled, the newly calculated graphical display can be shown on the monitor.

Within the scope of the invention it is not mandatory for the aforementioned steps to be executed in the above-described sequence. In a further embodiment, the method steps can be interleaved so that in the control of the device a gesture is in turn picked up via acceleration data, which in turn brings about a control.

In addition, it is possible for individual portions of the above-described method to be embodied as individually marketable units and the remaining portions of the method to be embodied as different marketable units. Hence, the method according to the invention can be executed as a distributed system on different computer-based facilities (for example client-server facilities). It is possible, for example, for the portable controller to have different submodules implemented partially on the controller and partially on the conversion module and/or partially on other computer-based facilities. This greatly increases the flexibility and the field of application of the solution according to the invention.

In a further embodiment of the invention, the portable controller is merged with the conversion module and integrated to form one component. This component then has a wireless interface to the device control system (or in particular the respective application, such as the image display application).

The above-described object is also achieved by a non-transitory, computer-readable storage medium encoded with programming instructions, which can be loaded directly into a memory of the control computer of a medical device. The program code or instructions cause the computer to execute all the steps of the method as described above.

The computer-readable medium can be an electronically readable data carrier, for example a DVD, a magnetic tape or a USB stick on which electronically readable control information, in particular software, is stored.

### BRIEF DESCRIPTION OF THE DRAWINGS

FIG. 1 schematically illustrates a control system according to a preferred embodiment of the invention.

FIG. 2 schematically illustrates a portable control unit according to a preferred embodiment of the invention.

FIG. 3 is a top view of the portable controller according to a preferred embodiment of the invention.

FIG. 4 is a top view of the portable controller according to an alternative embodiment of the invention.

FIG. 5 is a flowchart of a method according to a preferred embodiment of the invention.

### DESCRIPTION OF THE PREFERRED EMBODIMENTS

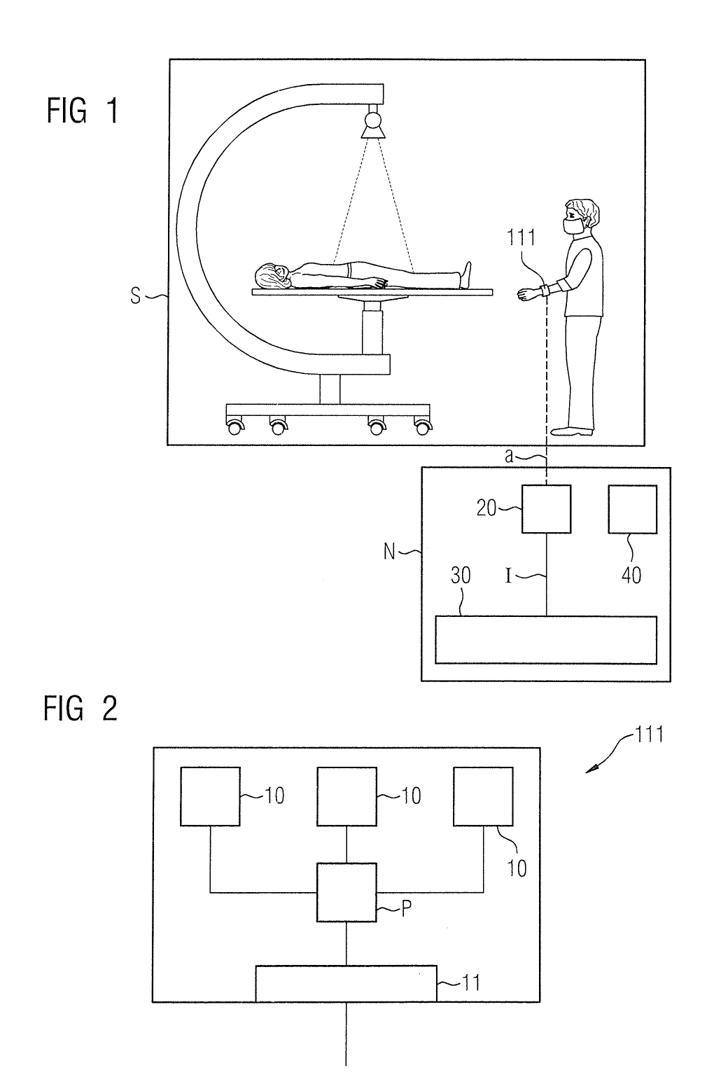

FIG. 1 is a schematic illustration of an environment for the control system according to the invention. The control system is operated in the sterile environment of an operating theater or an interventional medical facility. This is divided into a sterile environment S and a non-sterile (normal) environment N. The sterile environment S is subject to requirements with respect to sterility.

It is nevertheless necessary for control measures to be implemented in the sterile environment of the operating theater, such as, for example, in conjunction with the acquisition and display of medical images, such as X-ray images, CT images, MRI images or images from other modalities that have to be used in advance of and also during a surgical intervention.

The common method of control by means of a keyboard or a monitor (touchscreen) cannot be used since the keyboard and monitor are either not located in the sterile environment S or cannot be operated directly and with the necessary precision due to the corresponding coverings.

To increase the precision of the control measures, the invention provides a contactless control system based on gestures of the user in the sterile environment S, wherein the gestures are acquired via a portable controller 111. The portable controller 111 can be embodied as a circlet or ring and has one or more inertial sensors 10, which can be acceleration sensors or gyro sensors, and are designed to record acceleration data for a body part of the user (arm or hand, finger or foot etc.).

The acquired acceleration data are forwarded via a wireless interface 11 to a conversion module 20, which is configured to receive the transmitted acceleration data and convert it into instructions I, the instructions I being in a format to control the respective medical device 30. The device 30 can be, for example, a display monitor for displaying the radiological image data, wherein the image data are displayed with changes in response to a user input. In one embodiment of the invention, the conversion module 20 is embodied as a component that is separate from the portable controller 111 and spaced apart therefrom. In an alternative embodiment of the invention, the conversion module 20 can also be implemented on a (further) computer on which the program is executed for the control of the device or the image display.

A first gesture can be assigned, for example, to a first instruction in order to control a function in order to display a specific area as enlarged in an image and with more detailed information. A second gesture can be assigned, for example, to a second instruction in order to control a function in order to select certain images. A third gesture can be assigned, for example, to a third instruction in order to control a function in order to display the images in a different perspective. In a preparatory phase, it is possible to define different gestures and to assign at least one instruction for the control. Advantageously, certain gestures and gesture-instruction assignments, such as, for example, an intuitive to-and-fro movement of the hand can be used to scroll through a stack of images.

The sterile environment S contains the other devices commonly present, which for purposes of clarity are not shown in FIG. 1 and are also controlled by another preferably further portable controller 111. Advantageously, it is also possible for devices located in the non-sterile normal environment N or a different position to be controlled from the sterile environment S. The normal environment can contain a display device or a monitor 40 on which confirmation signals are acquired and/or radiological images are displayed. The display device 40 can also be located in the sterile environment S and controlled by the contactless control system according to the invention by the portable controller (this case is not shown in FIG. 1).

FIG. 2 is a schematic illustration of the structure of the portable controller. As a rule, it comprises a plurality of sensors 10 with which the acceleration of the body part to which they are attached can be measured. It is also possible for a processor P to be provided in order to forward the signal picked up at the sensors to a wireless interface 11 which is intended to transfer the acceleration data to the conversion module 20 which is commonly assigned to the control system as a separate component and can also be located in the normal environment N or at least partially integrated in the device 30 which is to be controlled.

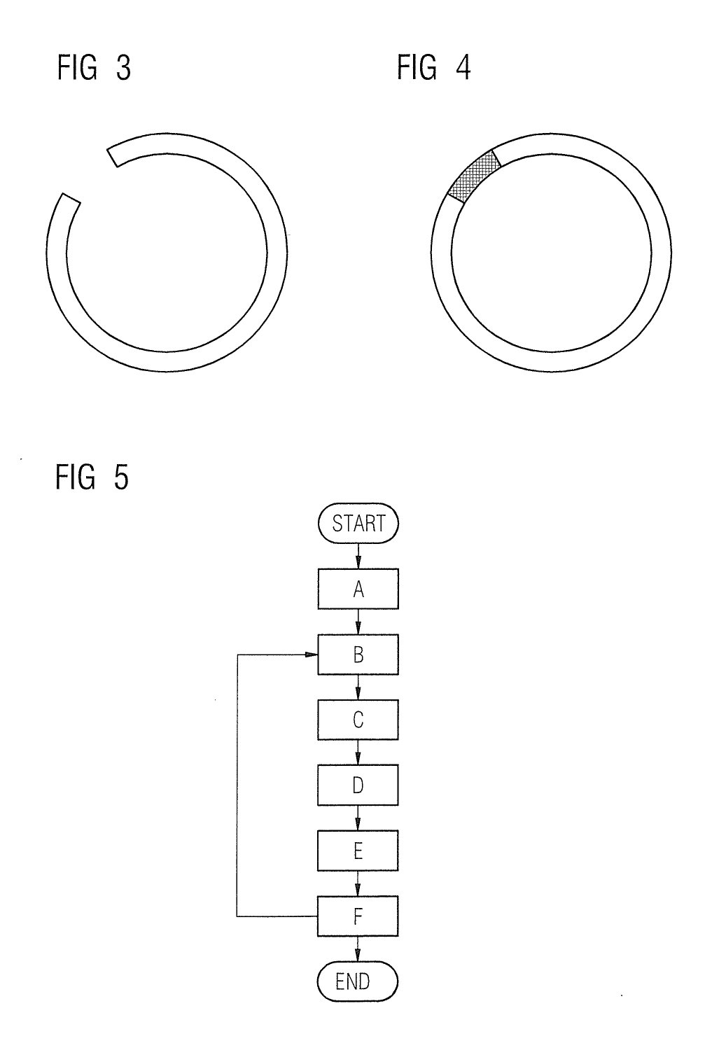

The portable controller 111 is designed such that it can be slipped quickly and easily over an extremity of the user. To this end, it is embodied as an open ring as represented schematically in FIG. 3. The opening renders the ring expandable and widening the opening enables its shape to be easily changed for mounting or removal in order, in pushed-on or mounted state, to create a clamping effect and enclose the body part such that permanent positioning can be ensured and the possibility of slipping can be reliably avoided.

In an alternative embodiment of the invention, the portable controller 111 is designed as a closed ring and is composed of two different materials: a first material with a slip-free surface (for example a rubber-like material) and a second material, which is expandable. The result is that it is still possible for the radius of the circumferentially closed ring to be changed, thus facilitating the mounting and removal of the ring. This embodiment of the invention is shown in FIG. 4. The two different materials are indicated by dashed lines. It is also possible to create the control unit in an annular shape as a closed or interrupted (open) ring and from a material which is flexible per se and permits temporary extension.

The following describes the course of the control method in more detail with reference to FIG. 5. After the start, in step A an activation signal is acquired to indicate that the contactless control is to be activated and that the inertial sensors 10 are to acquire acceleration data in a predefinable period. Then, the acceleration data are acquired in step B and transmitted in step C via the WLAN interface (for example based on the standard for the IEEE-802.11 family) or another wireless interface or radio link to the conversion module 20. When the acceleration data have been received in step D on or at the conversion module 20, it is converted in step E into instructions I. The device 30 is then controlled on the basis of these instructions I (step F). After this, the method can be repeated from step B or is terminated.

Although modifications and changes may be suggested by those skilled in the art, it is the intention of the inventors to embody within the patent warranted hereon all changes and modifications as reasonably and properly come within the scope of their contribution to the art.

We claim as our invention:

1. A control system for controlling a medical device while observing sterile conditions comprising:
a portable controller that is temporarily attachable to a body part of a user for co-movement with movement of said body part;
said portable controller comprising at least one inertial sensor that acquires acceleration data during movement of said body part;
a conversion module;
said portable controller comprising a wireless interface that transmits the acquired acceleration data to said conversion module; and
said conversion module being configured to receive the transmitted acceleration data and to convert the received acceleration data into instructions having a format adapted to control said medical device.

2. A control system as claimed in claim 1 wherein said inertial sensor comprises at least one sensor selected from the group consisting of acceleration sensors and gyro sensors.

3. A control system as claimed in claim 1 wherein:
said control module is configured to operate in either of a confirmation mode and a direct mode;
said control module in said confirmation mode is configured to require confirmation of an instruction, by a confirmation signal, before converting the received acceleration data into said instruction; and
said control module in said direct mode is configured to convert the received acceleration data into said instruction directly, without a confirmation signal.

4. A control system as claimed in claim 1 comprising:
a display monitor in communication with said conversion module to receive said instruction from said conversion module and to visually display a representation of said instruction;
and wherein said control module is configured to operate in a confirmation mode in which said instruction must be confirmed by a confirmation signal before the received acceleration data are converted into said instruction;
a computer in communication with said control module and with said display monitor, said computer being configured to cause a switching element to be shown on said display monitor; and
said control module, via said computer, being configured to switch said switching element in response to a user gesture acquired by said inertial sensor so as to confirm a preceding instruction and thereby initiate conversion of the received acceleration data into said preceding instruction.

5. A control system as claimed in claim 1 wherein said control module is configured to operate in a confirmation mode which requires receipt of a confirmation signal by said control module before converting the received acceleration data into said instruction, and said control system further comprising an actuatable switch in communication with said conversion module that, when activated, emits a confirmation signal that confirms a preceding instruction and causes said control module to initiate conversion of the received acceleration data into said preceding instruction.

6. A control system as claimed in claim 5 wherein said switch is selected from the group consisting of mechanical switches and voice-controlled switches.

7. A control system as claimed in claim 1 wherein said control module is configured to be activated dependent on acceleration data acquired by the inertial sensor and transmitted to the control module that represents an activation signal that, when received by said conversion module, activates said conversion module for a predetermined time duration, and by acceleration data acquired by the inertial sensor and transmitted to the conversion module that represent a deactivation signal that deactivates said conversion module.

8. A control system as claimed in claim 1 comprising a display monitor and a computer in communication with said display monitor and with said conversion module, said computer being configured to display a user interface at said display monitor that includes displayed elements for additionally controlling said medical device, and wherein said displayed elements are activatable in a contact-free manner.

9. A control system as claimed in claim 8 wherein said control elements are activated by a predetermined user gesture detected by said inertial sensor and transmitted to said conversion module, that initiates or terminates at least one control function designated by the respective control element.

10. A control system as claimed in claim 1 wherein said portable controller is designed to be worn at an arm, finger or wrist of the user.

11. A control system as claimed in claim 10 wherein said portable controller is an armband, a ring or a watch.

12. A control system as claimed in claim 1 wherein said conversion module is integrated with said portable controller.

13. A control system as claimed in claim 1 wherein said inertial sensor acquires said acceleration data independently of a position of the user and during a change in position of the user.

14. A control system as claimed in claim 1 comprising an auxiliary module that provides an acknowledgement to the user of successful conversion of said instruction.

15. A control system as claimed in claim 14 wherein said auxiliary module is a vibration module that emits a vibration that is perceptible by the user upon said successful conversion of the instruction.

16. A method for controlling a medical device while observing sterile conditions, comprising:
temporarily attaching a controller, in a sterile environment, to a body part of a user so that said controller is co-movable with movement of the body part;
activating the controller in the sterile environment;
after activation of the controller, acquiring acceleration data of said body part with said controller;
wirelessly transmitting the acquired acceleration data to a conversion module;
in said conversion module, converting the received acceleration data into an instruction; and
controlling said medical device dependent on said instruction.

17. A method as claimed in claim 16 comprising:
in addition to acquiring said acceleration data, acquiring data representing an angular velocity of the body part of the user with said controller.

18. A method as claimed in claim 17 comprising controlling images produced by said medical device at a display monitor dependent on said instruction.
